(19) United States
(12) Patent Application Publication
(10) Pub. No.: US 2019/0127587 A1
(43) Pub. Date: May 2, 2019

(54) SO2 Containing Dispersion With High Salt Stability

(71) Applicants: Wolfgang Lortz, (US); Ulrich FISCHER, Mombris (DE); Christian PANZ, Wesseling-Berzdorf (DE); Gabriele BERGMANN, Grosskrotzenburg (DE)

(72) Inventors: Wolfgang Lortz, (US); Ulrich FISCHER, Mombris (DE); Christian PANZ, Wesseling-Berzdorf (DE); Gabriele BERGMANN, Grosskrotzenburg (DE)

(21) Appl. No.: 15/743,214

(22) PCT Filed: Jun. 28, 2016
(86) PCT No.: PCT/EP2016/064994

§ 371 Date: Jan. 9, 2018

(30) Foreign Application Priority Data
Jul. 10, 2015 (EP) 15176284.6

(51) Int. Cl. C09C 1/40; C09D 7/62; C09D 17/00; C11D 3/12; C11D 11/00; C11D 17/00; D21H 19/40; A61K 8/26; A61K 8/893; A61K 8/894; A61Q 19/00; A61K 9/51; A61K 8/02; A61K 8/06; A61K 9/107

(52) U.S. Cl. CPC C09C 1/405; C01P 2004/62; C09D 17/001; C09D 17/004; C11D 3/128; C11D 11/0023; C11D 17/0013; D21H 19/40; A61K 8/26; A61K 8/893; A61K 8/894; A61Q 19/00; A61K 9/5115; A61K 9/5146; A61K 8/0241; A61K 8/062; A61K 9/107; C01P 2004/50; C01P 2004/64; C09D 7/62

(57) ABSTRACT

An aqueous dispersion comprising a surface-modified hydrophilic mixed oxide powder comprising silicon and aluminum and water, characterized in that

a) the surface of the particles has Si and Al atoms and
b) the surface modification has an Si atom bonded to a hydrocarbon radical via a C atom, and
c) the carbon content of the surface-modified mixed oxide powder is 3-25% by weight.

The invention relates to SiO₂ containing dispersions with high salt stability and to the preparation and use thereof.

Improving the stability of aqueous silicon dioxide dispersions is a subject of research. Attempts are commonly made to protect the dispersion from sedimentation and reagglomeration by providing the silicon dioxide particles with appropriate surface modification.

Thus, for example, US2004241101 discloses a stable pharmaceutical dispersion which contains silicon dioxide particles surface-modified with polyethylene glycols. The latter may be obtained, for example, by reacting an ammonia-stabilized colloidal silicon dioxide with a polyethoxylated trialkoxysilane.

US2002172827 is concerned inter alia with the production of redispersible, nanoscale silicon dioxide particles. This involves coating a negatively charged silica sol with an aluminum oxide. Sodium dodecylbenzenesulfonate is then added as a surface-modifying agent.

WO2004035474 claims a process for producing a stable aqueous dispersion obtained by mixing silanized, colloidal silicon dioxide particles with an organic binder. The silanizing agent is for example a glycidylepoxysilane. The organic binder may be a polyethylene glycol.

In Part. Syst. Charact. 2014, 31, 94-100 colloidal silicon dioxide particles are surface-modified with 2-[methoxy(polyethyleneoxy)propyl]trimethoxysilane to increase salt stability. Clean Technology, www.ct-si.org, ISBN 978-1-4398-3419-0 (2010) 25-28 also addresses salt stability.

WO03/106339 describes a precipitated silica having a BET surface area of 150-400 m²/g, a CTAB surface area of 140-350 m²/g and an Al₂O₃ content of 0.2-5 wt %. This silica may be modified with a multiplicity of silanes and result in both hydrophilic and hydrophobic products. The ratio of silane to precipitated silica may also be varied within wide limits, namely 0.5 to 50 parts of silane based on 100 parts of precipitated silica. The reaction may be effected in the dispersion of the precipitated silica with subsequent drying and heat treatment. Conditions in this regard are not recited and the properties of the dispersion are not further specified.

WO02/22745 discloses a process for primer coating of steel in which an aqueous aluminum oxide-silicon dioxide sol comprising 0.05-2.0 wt % of aluminum oxide is employed.

The aluminum oxide-silicon dioxide sol may contain a silane coupling agent having alkoxysilane groups and an organic radical having a functional group, such as an amino, epoxide or isocyanate.

WO2010/042672 discloses a coating composition for thermoplastic and thermosetting substrates, comprising an aqueous dispersion having a pH of less than 7.5. Said composition contains surface-modified silicon dioxide nanoparticles having a median particle diameter of 40 nm or less, an alkoxysilane oligomer and a silane coupling agent. Suitable surface-modifying agents are those having a radical that can react with the silanol groups on the silicon dioxide surface and having a hydrophilic radical, for example an acid radical, an ammonium radical, a polyoxyethylene radical or a hydroxyl group.

However it has been found that for many applications the salt stability achieved is insufficient. It is accordingly an object of the present invention to provide a dispersion having improved salt stability. It is a further object of the invention to provide a process for producing this dispersion.

The invention provides an aqueous dispersion comprising a surface-modified hydrophilic mixed oxide powder comprising silicon and aluminum and water, wherein

a) the surface of the particles has Si and Al atoms and
b) a surface modification has an Si atom bonded to a hydrocarbon radical via a C atom and
c) the carbon content of the surface-modified mixed oxide powder is 3-25% by weight.

The aqueous dispersion is defined as being composed of solid and liquid phases. The liquid phase may comprise dissolved constituents of air.

It is an essential feature of the invention that the surface of the particles has Si and Al atoms. Dispersions comprising particles which prior to surface modification have an aluminum oxide shell, i.e. where the surface has only aluminum atoms, or those composed only of aluminum oxide exhibit lower stabilities than the aqueous dispersions according to the invention.

The term “mixed oxide powder” is to be understood as meaning an intimate mixture of the mixed oxide components aluminum and silicon on an atomic level where the particles may also have Si—O—Al bonds.

“Surface-modified” is to be understood as meaning that the silica on its surface bears groups which very largely give the particles the hydrophilic properties exhibited by the unmodified silica. This causes the aqueous dispersion to remain stable. The term “stable” is to be understood as meaning that no appreciable re-agglomeration, and thus no sedimentation, occurs. In an aqueous solution hydrophobized particles would reagglomerate and separate in a very short time.

In the case of the aqueous dispersion of the present invention, a 0.5 weight percent aqueous dispersion is stable in a reference solution simulating sea water for at least one month at a temperature of 60° C. The stability is tested in a reference solution which is obtained by adding sufficient fully demineralized water at 23° C. to a mixture of 28.500 g of NaCl, 0.220 g of NaHCO₃, 4.066 g of Na₂SO₄, 1.625 g of CaCl₂×2 H₂O, 3.162 g of MgCl₂×6 H₂O, 0.024 g of SrCl₂×6 H₂O and 0.721 g of KCl to give 1000 ml of solution.

In a particular embodiment the mixed oxide powder, where the Si atom which is bonded to a hydrocarbon radical via a C atom, additionally has —Si—O—Al bonds, the Al atom being a constituent of the particle surface.

A suitable analytical method for detecting the surface elements and their bonds is X-ray-induced photoelectron spectroscopy (XPS). A depth profile may be generated by stepwise sputter etching. Additional information about the composition of the surface can be determined via energy-dispersive x-ray radiation (TEM-EDX). The composition of the total particle may be determined by chemical or physicochemical methods, for example x-ray fluorescence analysis.

The proportions of Al and Si in the surface-modified mixed oxide powder may be varied within wide limits. The zeta potential of the dispersion has proven a suitable measure. This zeta potential should be negative.

Expressed as a weight ratio of the oxides, an Al₂O₃/SiO₂ weight ratio is <1, i.e. Al₂O₃ is the secondary component at less than 50% by weight. It is been found that a mixed oxide powder comprising far lower proportions of aluminum oxide results in a better stability of the aqueous dispersion than a mixed oxide powder comprising high proportions of aluminum oxide. Low proportions is understood to mean that the Al₂O₃/SiO₂ weight ratio in the surface-modified mixed oxide powder is 0.1:99.9-5:95, particularly preferably 0.2:99.8-3:97.

The Al₂O₃/SiO₂ weight ratio at the surface may be greater, smaller or equal to the weight ratio in the total particle. An (Al₂O₃/SiO₂)_{surface}/(Al₂O₃/SiO₂)ₜₜₗ ratio of 0.1-10 is preferred. “ttl.” corresponds to the weight ratio in the total particle. The weight ratio on the surface may be determined for example by X-ray-induced photoelectron spectroscopy (XPS). The weight ratio in the total particle may be determined by chemical or physicochemical methods, for example X-ray fluorescence analysis.

It is preferable when mixed oxide particles obtained from pyrogenic processes are present. In these processes, silicon and aluminum compounds are reacted in a flame generated by the reaction of hydrogen and oxygen. The thus obtained powders are referred to as “pyrogenic” or “fumed”. The reaction initially forms highly disperse primary particles, which in the further course of reaction coalesce to form aggregates. The aggregate dimensions of these powders are generally in the range of 0.2-1 μm. Said powders may be converted into the nm range advantageous for the present invention by suitable grinding and subsequently treated with a surface-modifying agent.

It has been found that the best results in terms of salt and temperature stability of the aqueous dispersion are obtained with a surface-modified mixed oxide powder which in the dispersion has a median particle diameter d₅₀ of 40-200 nm. The median particle diameter may be determined with the customary methods of light scattering for the determination of particle size distributions in dispersions known to those skilled in the art.

The surface-modified mixed oxide powder may be in the form of isolated individual particles and/or in the form of aggregated particles. In the case of aggregated particles the median particle diameter refers to the dimension of the aggregate.

The surface-modified mixed oxide present in the aqueous dispersion according to the invention is inter alia characterized in that the surface modification comprises a hydrocarbon radical attached via a C atom to an Si atom. The hydrocarbon radical is to be chosen such that the surface-modified mixed oxide exhibits hydrophilic properties in the aqueous dispersion. This depends for example on the number of carbon atoms in the hydrocarbon radical and on the presence of functional hydrophilic groups, such as hydroxyl, ether, amine or carboxyl groups. The hydrocarbon radical is preferably interrupted by one or more heteroatoms. With particular preference the heteroatom is O or N.

It is preferable when a surface modification is selected from the group consisting of Si—(CH₂)ₙ—Yₘ—R, wherein Si is the Si atom bonded to a hydrocarbon radical via a C atom and

n=1, 2 or 3 and m=0 or 1;
R is a radical which does not impart hydrophobic properties and preferably
in the case where m=1
R═—H, —CH₃, —C₂H₅, —OH, —OCH₃, —OC₂H₅, —C(═O)OCH₃, —C(═O)OC₂H₅, —O—C(═O)CH₃, —O—C(═O)CH₃, —O—C(═O)CH═CH₂, —O—C(═O)CH═CH(CH₃), —C(═O)CH₃, —C(═O)H, NH_{2;}

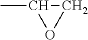

and in the case where m=0, R represents the abovementioned radicals but without —H, —CH₃, —C₂H₅. Y═—(OCR¹R²—CR³R⁴)ₒ—, o=1-30, R¹, R², R³, R⁴=independently of one another H or CH₃, particularly preferably o=5-15 and R¹, R², R³, R⁴═H;
—(OCR¹R²—CR³R⁴—CR⁵R⁶)ₚ—, p=1-30, R¹, R², R³, R⁴, R⁵, R⁶=independently of one another H or CH₃, —NHCH₂CH₂O—, —NH(CH₂)₂NH(CH₂)₂—, —NH(CH₂)₂NH(CH₂)₂—.
   or is a mixture of the aforementioned radicals R and Y.

It is likewise conceivable for Y to comprise branched polyethylene glycols. Here, R and at least one of the R¹-R⁶ radicals represents an —(OCH₂—CH₂)ᵣ moiety where r=5-15.

In the aqueous dispersion according to the invention the proportion of water is preferably 50-90 wt % and of surface-modified mixed oxide powder is preferably 10-50 wt %. Depending on the planned further use, the proportion of surface-modified mixed oxide powder may be reduced further.

It has been found that the liquid phase may also contain small proportions of alcohol, such as methanol, ethanol, propanol or butanol, in addition to water. The proportion of alcohol is generally less than 10% by weight, preferably 3-7% by weight, in each case based on the dispersion.

The pH of the liquid phase of the aqueous dispersion is preferably 8-12, particularly preferably 9-11.

The aqueous dispersion according to the invention may comprise small amounts, less than 100 ppm, of customary dispersants. However, the presence of dispersants is not desired in the context of the present invention. The stabilizing effect of the aqueous dispersion according to the invention derives solely from the surface-modified mixed silicon-aluminum oxide powder.

The invention further provides a process for producing the dispersion in which

a mixed oxide powder comprising silicon and aluminum, which has Si atoms and Al atoms each bearing hydroxyl groups on the surface of the particles, is dispersed in an aqueous solvent and subsequently
an agent for surface modification is added in which an Si atom is bonded to a hydrocarbon radical via a C atom and the Si atom is further bonded to one or more hydroxyl groups, alkoxy groups, halide groups or mixtures thereof, and
the mixture is caused to react and the hydrolysis product is optionally separated and the pH is optionally adjusted,
wherein the agent for surface modification is added in such an amount as to obtain a carbon content of 3-25% by weight, taking account of the hydroxyl groups, alkoxy groups or halide groups eliminated in the hydrolysis.

When compounds having alkoxy groups are employed the hydrolysis product is an alcohol, for example methanol or ethanol.

Numerous methods of dispersing are available to those skilled in the art. To produce finely divided dispersions, apparatuses such as for example ultrasound probes, ball mills, stirred ball mills, rotor/stator machines, planetary kneaders/mixers or high-energy mills or combinations thereof are available. Thus for example a preliminary dispersion may be prepared using a rotor/stator system which in a subsequent step is subjected to further milling by means of a high-energy mill. This combination makes it possible, for example, to produce extra fine dispersions having a particle diameter of 200 nm or less. In the case of a high-energy mill, a preliminary dispersion under high pressure is divided into two or more streams, which are then decompressed through a nozzle and impinge exactly on one another.

It has been proven advantageous to proceed directly from an aqueous dispersion of a silicon- and aluminum-comprising mixed oxide powder.

The mixture is generally reacted by adjusting the pH to 11 or more, thermally treating the mixture at a temperature of 50-95° C. over a period of 1-30 minutes, and then optionally adjusting the pH to 8-10.

The mixed silicon-aluminum oxide powder employed in the process according to the invention is one where Si atoms and Al atoms each bearing hydroxyl groups are present at the surface of the particles.

One particular embodiment employs a fumed mixed silicon-aluminum oxide powder. Commercially available fumed mixed silicon-aluminum oxide powders are AEROSIL® MOX 80 having a BET surface area of 60-100 m²/g and an aluminum oxide content of 0.3-1.3% by weight and AEROSIL® MOX 170 having a BET surface area of 140-200 m²/g and an aluminium oxide content of 0.3-1.3% by weight, both from Evonik Industries. Both fumed mixed silicon-aluminum oxide powders may be employed with preference. AEROSIL® MOX 170 is particularly preferred.

It is additionally possible to employ the fumed mixed silicon-aluminum oxide powder disclosed in EP-A-995718. Said powder is obtained by reacting a vaporous silicon dioxide precursor and an aluminum chloride solution in a flame. The fine distribution of aluminum chloride in the aerosol and during the genesis of the oxide in the gas phase results in substantially homogeneous incorporation of the aluminum.

It is likewise possible to employ the fumed mixed silicon-aluminum oxide powder disclosed in EP-A-2500090 where the weight ratio of (Al₂O₃/SiO₂)ₜₜₗ in the overall particle is 0.002 to 0.05, and the (Al₂O₃/SiO₂)_{surface} weight ratio of the particles in a layer close to the surface is lower than in the overall particle. The aluminum oxide concentration at the surface has thus been reduced further.

For the process of the invention, the agent for surface modification is selected preferably from the group consisting of X₄₋ₐ[Si—(CH₂)ₙ—Yₘ—R]ₐ where

a=1, 2 or 3; preferably a=1; n=1, 2 or 3; m=0 or 1,
X═H, OH, OCH₃, OC₂H₅, OCH₂CH₂H₃, OCH(CH₃)₂; Cl,
R is a radical which does not impart hydrophobic properties and preferably
in the case where m=1
R═—H, —CH₃, —C₂H₅, —OH, —OCH₃, —OC₂H₅, —C(═O)OCH₃, —C(═O)OC₂H₅, —O—C(═O)CH₃, —O—C(═O)CH₃, —O—C(═O)CH═CH₂, —O—C(═O)CH═CH(CH₃), —C(═O)CH₃, —C(═O)H, NH_{2;}

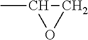

and in the case where m=0, R represents the abovementioned radicals but without —H, —CH₃, —C₂H₅.
Y═—(OCR¹R²—CR³R⁴)ₒ—, o=1-30, R¹, R², R³, R⁴=independently of one another H or CH₃, particularly preferably o=5-15 and R¹, R², R³, R⁴═H;
   —(OCR¹R²—CR³R⁴—CR⁶R⁶)ₚ—, p=1-30, R¹, R², R³, R⁴, R⁵, R⁶=independently of one another H or CH₃, —NHCH₂CH₂O—, —NH(CH₂)₂NH(CH₂)₂—, —NH(CH₂)₂NH(CH₂)₂—.
      or is a mixture of the abovementioned radicals R and Y.

It is likewise conceivable for Y to comprise branched polyethylene glycols. Here, R and at least one of the R¹-R⁶ radicals represents an —(OCH₂—CH₂)ᵣ moiety where r=5-15.

With particular preference, the agent for surface modification may be selected from the group consisting of (CH₃O)₃Si(CH₂)₃—OCH₃, (CH₃O)₃Si(CH₂)₃—(OCH₂CH₂)₃—OCH₃, (CH₃ O)₃Si(CH₂)₃—(OCH₂CH₂)₆₋₉—OCH₃, (CH₃ O)₃Si(CH₂)₃—(OCH₂CH₂)₉₋₁₂—OCH₃, (CH₃ O)₃Si(CH₂)₃—(OCH₂CH₂)₂₁₋₂₄—OCH₃ and (CH₃CH₂ O)₃Si(CH₂)₃—(OCH₂CH₂)₈₋₁₂OH.

The agent for surface modification may further be selected from the group consisting of (RO)₃Si—(CH₂)₃—NH₂, (RO)₃Si—(CH₂)₃—CH—CH₂—NH₂, (RO)₃Si—(CH₂)₃—NH—(CH₂)₂—NH₂, (RO)₃Si—(CH₂)₃—NH—(CH₂)₂NH(CH₂)—NH₂, (RO)₃Si—(CH₂)₃—N—[(CH₂)₂NH(CH₂)—NH₂]₂, R═CH₃, C₂H₅.

Additionally suitable for the surface modification are aqueous compositions which comprise organopolysiloxanes having glycidyl ether alkyl radicals, acryloyloxyalkyl radicals and/or methacryloyloxyalkyl radicals. Furthermore, as further functional groups, the organopolysiloxane may comprise aminoalkyl radicals or alkyl radicals or aminoalkyl and alkyl radicals.

Each silicon atom in the organopolysiloxane preferably bears a functional group. The organopolysiloxane-containing compositions may be obtained by mixing water-soluble organosilanes of the formula I

H₂N(CH₂)_{f}(NH)_{g}(CH₂)ᵢ—Si(CH₃)ₕ(OR)₃₋ₕ (I),

where 0≤f≤6, g=0 if f=0, g=1 if f>1, 0≤i≤6, 0≤h≤1 and R is a methyl, ethyl, propyl or isopropyl group, preferably aminopropyltriethoxysilane,
with
water-soluble organosilanes of the formula II which, however, are not stable in the aqueous medium

X—CH₂O(CH₂)₃—Si(CH₃)ₕ(OR)₃₋ₕ (II),

where 0≤h≤1 and R is a methyl, ethyl, propyl or isopropyl radical, preferably glycidyloxypropyltrimethoxysilane, and

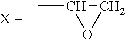

and/or
organosilanes of the formula III

H₂C═CR′—COO(CH₂)₃—Si(CH₃)ₕ(OR)₃₋ₕ (III),

where 0≤h≤1, R is a methyl, ethyl, propyl or isopropyl radical and R′ is a methyl or hydrogen radical, preferably methacryloxypropyltrimethoxysilane,
and non-water-soluble organosilanes of the formula IV

R″—Si(CH₃)ₕ(OR)₃₋ₕ (IV),

where 0≤h≤1, R is a methyl, ethyl, propyl or isopropyl radical and R″ is a linear, branched or cyclic hydrocarbon radical having 1 to 8 C atoms, preferably propyltrimethoxysilane,
in the molar ratio M=a/(b+c+d), where a is the sum of the number of moles of the organosilanes of formula I, b is the sum of the number of moles of the organosilanes of formula II, and c is the sum of the number of moles of the organosilanes of formula III, and d is the sum of the number of moles of the organosilanes of formula IV, where 0≤M≤3 and at least b>0 or c>0.

The mixture is admixed with a water/acid mixture, the pH of the reaction mixture is adjusted to a value between 1 and 8, and the alcohol is removed.

In idealized form, the organopolysiloxane-containing compositions can be represented according to the formula

HO[Si(A*)(OH)_{z}(CH₃)_{1-z}O]ₐ[Si(B*)(OH)_{y}(CH₃)_{1-y}O]_{b}[Si(C*)(OH)_{w}(CH₃)_{1-w}O]_{c}[Si(D*)(OH)ᵥ(CH₃)₁₋ᵥO]_{d}H(HX)ₑ (V)

where A* is an aminoalkyl radical derived from the formula I,
B* is a glycidyl ether alkyl radical derived from the formula II,
C* is an acryloyloxyalkyl or methacryloyloxyalkyl radical derived from the formula III, and
D* is an alkyl radical according to the general formula IV,
HX is an acid, where X is an inorganic or organic acid radical,
v is 0 or 1 and w is 0 or 1 and y is 0 or 1 and z is 0 or 1 and a+b+c+d≥4 and a≤e≤2 a, where 0≤a/(b+c+d)≤3.

The organopolysiloxane-containing compositions preferably have a pH of 1-8, more preferably of 3-6.

A readily redispersible, surface-modified powder can be obtained from the aqueous dispersion of the invention by removal of the liquid phase, by means of spray drying, for example. This powder can be incorporated into an aqueous phase with a low input of energy, for example by stirring, without appreciable aggregation of the particles. The median particle diameter d₅₀ in this aqueous dispersion may be 40-200 nm.

The invention further provides an aqueous dispersion obtainable by the process according to the invention.

The invention further provides a surface-modified mixed oxide powder comprising silicon and aluminum obtainable by

dispersing a mixed oxide powder comprising silicon and aluminum, which has Si atoms and Al atoms each bearing hydroxyl groups on the surface of the particles, in an aqueous solvent and subsequently adding an agent for surface modification in which an Si atom is bonded to a hydrocarbon radical via a C atom and the Si atom is further bonded to one or more hydroxyl groups, alkoxy groups, halide groups or mixtures thereof, and
causing the mixture to react and
separating the hydrolysis product.

The invention further provides therefore a surface-modified mixed oxide powder comprising silicon and aluminum, in which

a) the Al₂O₃/SiO₂ weight ratio is 0.1:99.9-5:95,
b) (Al₂O₃/SiO₂)_{surface}/(Al₂O₃/SiO₂)ₜₜₗ is 0.1-10, and
c) the carbon content thereof is 3-25% by weight,
d) it has a surface modification in which an Si atom is bonded to a hydrocarbon radical via a C atom.

The BET surface area of the surface-modified mixed oxide powder is preferably 40-500 m²/g, particularly preferably 80-300 m²/g. The BET surface area is determined according to DIN 66131.

The invention further provides for the use of the aqueous dispersion according to the invention and of the surface-modified mixed oxide powder comprising silicon and aluminum according to the invention, in each case as a constituent of pharmaceutical preparations, cosmetic preparations, water-based paints and coatings, of cleaning products, of dishwashing detergents and of coloured coating slips in the paper industry.

### EXAMPLES

Salt Stability at Elevated Temperature

28.500 g of NaCl, 0.220 g of NaHCO₃, 4.066 g of Na₂SO₄, 1.625 g of CaCl₂×2 H₂O, 3.162 g of MgCl₂×6 H₂O, 0.024 g of SrCl₂×6 H₂O and 0.721 g of KCl are dissolved in 900 g of deionized water (DI water) and the solution made up to 1 liter with DI water.

99.5 g of this solution are initially charged into a 125 ml wide-necked bottle made of NALGENE® FEP (tetrafluoroethylene-hexafluoropropylene copolymer; Thermo Scientific), 0.5 g of the dispersion under test is added and the mixture is homogenized by shaking. The mixture is stored in a drying cabinet at 60° C. and the occurrence of a precipitate is visually monitored.

### Input Materials

Mixed silicon-aluminum oxide

### A: AEROSIL® MOX 170, Evonik Industries

The powder has the following properties:

99% by weight silicon dioxide, 1% by weight aluminum oxide. The BET surface area is 173 m²/g.
(Al₂O₃/SiO₂)ₜₜₗ/(Al₂O₃/SiO₂)_{surface}=0.9.
B: In accordance with EP-A-995718, example 1. The powder has the following properties:
99.7% by weight SiO₂, 0.27% by weight Al₂O₃ content. The aluminum oxide content is distributed homogeneously. The BET surface area is 55 m²/g.

AERODISP® W 7512 S, Evonik Industries, is an acidic, low-viscosity, aqueous silica dispersion having a solids content of 12%. The solid on which it is based is AEROSIL® 200, Evonik Industries, a fumed silica having a BET surface area of 200 m²/g.

AERODISP® W 7520 N, Evonik Industries, is a low-viscosity, aqueous silica dispersion having a solids content of 20% which is stabilized with aqueous sodium hydroxide solution. The solid on which it is based is AEROSIL® 200, Evonik Industries, a fumed silica having a BET surface area of 200 m²/g.

AERODISP® W 630, Evonik Industries, is an aqueous aluminum oxide dispersion having a pH of 3-5 and a solids content of 30%. The solid on which it is based is AEROXIDE® Alu C, Evonik Industries, a fumed aluminium oxide having a BET surface area of 100 m²/g.

LUDOX® SM 30, Grace, is an aqueous, NaOH-stabilized, colloidal silica dispersion having a particle size of 8 nm and an SiO₂ content of 30 wt %.

LUDOX® HS 40, Grace, is an aqueous, NaOH-stabilized, colloidal silica dispersion having a particle size of 12 nm and an SiO₂ content of 40 wt %.

LUDOX® CL, Grace, is an aqueous dispersion of Al-coated, colloidal silica having a particle size of 22 nm. The pH is 3.5-4.5, the solids content 39-43 wt %.

Agents for Surface Modification

SM1: 2-[methoxy(polyethyleneoxy)₆₋₉propyl]trimethoxysilane
SM2: hydrolysate of 3-glycidyloxypropyltrimethoxysilane according to example 1, EP-A-832911

Water: deionized water is used; aqueous sodium hydroxide solution: 25% by weight NaOH; hydrochloric acid: 20% by weight HCl

### Example 1 (Inventive): Mixed Silicon-Aluminium Oxide A and SM1

### Production of a 20 Percent Dispersion of Mixed Silicon-Aluminum Oxide A

A 100 I stainless steel mixing vessel was initially charged with 37 kg of water. Subsequently, under shear conditions (Ystral Conti-TDS 3 (stator slots: 4 mm ring and 1 mm ring, rotor-stator gap about 1 mm), an initial 10 kg of AEROSIL® MOX 170 are aspirated. The remaining 5 kg were aspirated stepwise in amounts of about 1 kg each time. After addition was complete, the mixture was sheared at 3000 rpm for 30 min. To grind any residual proportions of coarse particles this predispersion was passed in two runs through a Sugino Ultimaizer HJP-25050 high-energy mill at a pressure of 2500 bar with diamond nozzles of 0.25 mm in diameter, thus subjecting it to further intensive grinding.

The median particle diameter d₅₀ determined by static light scattering (LA-950, Horiba Ltd., Japan) is 112 nm.

9.63 g of SM1 are added gradually to 40 g of this dispersion while stirring. There is an initial viscosity increase though this falls again upon further addition. The mixture is then adjusted to pH 11 with aqueous sodium hydroxide solution with stirring and the mixture is heated to 90° C. After 10 minutes at 90° C. the mixture is left to cool to room temperature and the mixture is adjusted to pH 9 with hydrochloric acid.

d₅₀=121 nm; stability in reference solution at 60° C.: 9 months.

### Example 2 (Inventive): Mixed Silicon-Aluminum Oxide B and SM2

A 20 percent dispersion of mixed silicon-aluminum oxide B is produced according to example 1. The median particle diameter d₅₀ determined by static light scattering (LA-950, Horiba Ltd., Japan) is 82 nm.

6.82 g of DYNALYSAN® HYDROSIL 2926 are added slowly to 40 g of this dispersion while stirring. The mixture now has a pH of 2.84. The pH is then adjusted to 11 with sodium hydroxide solution and the mixture heated to 90° C. for 10 min. After cooling, the pH is adjusted to 9 with hydrochloric acid.

d₅₀=93 nm; stability in reference solution at 60° C.: 1 month

### Example 3 (Comparative)

11.3 g of SM1 are added slowly with stirring to 67 g of AERODISP® W 7512 S. There is an initial viscosity increase though this falls again upon further addition. The mixture is then adjusted to pH 11 with aqueous sodium hydroxide solution with stirring and the mixture is heated to 90° C. After 10 minutes at 90° C. the mixture is cooled and adjusted to pH 9 with hydrochloric acid.

d₅₀=109 nm; stability in reference solution at 60° C.: 1 day

### Example 4 (Comparative)

11.3 g of SM1 are added slowly with stirring to 40 g of AERODISP® W 7520 N. The mixture is then adjusted to pH 11 with aqueous sodium hydroxide solution with stirring and the mixture is heated to 90° C. After 10 minutes at 90° C. the mixture is cooled and adjusted to pH 9 with hydrochloric acid.

d₅₀=99 nm; stability in reference solution at 60° C.: 1 day

### Example 5 (Comparative)

4.3 g of SM1 are added dropwise over 3 hours at 80° C. with stirring to 100 g of a LUDOX® 30 SM dispersion diluted with water to 10% by weight. The mixture is stirred at 80° C. for a further 6 hours. Stability in reference solution at 60° C.: 1 day

### Example 6 (Comparative)

30 g of SM1 are added to 249 g of LUDOX® HS 40. The dispersion is heated to 80° C. and stirred at this temperature for 16 hours.

Stability in reference solution at 60° C.: 1 day

### Example 7 (Comparative)

26.7 g of LUDOX® CL are diluted to 20% with 13.3 g of water. 13.0 g of SM1 are added to this sol slowly and with stirring. The mixture is then adjusted to pH 11 with aqueous sodium hydroxide solution with stirring and the mixture is heated to 90° C. After 10 minutes at 90° C. the mixture is cooled and adjusted to pH 9 with hydrochloric acid.

Stability in reference solution at 60° C.: 2 days

### Example 8 (Comparative)

26.7 g of AERODISP® W 630 are diluted to 20% with 13.3 g of water. 5.67 g of SM1 are added to this dispersion slowly and with stirring. The pH is then adjusted to 11 with aqueous sodium hydroxide solution with stirring and the mixture heated to 90° C. After 10 minutes at 90° C. the mixture is cooled and adjusted to pH 9 with hydrochloric acid.

Stability in reference solution at 60° C.: 1 day

The dispersions according to the invention of examples 1 and 2 exhibit very good salt stability at elevated temperatures. This stability is not present for comparative examples 3-8. Fumed silicas are used in examples 3 and 4, colloidal silica sols in examples 5 and 6, an Al-coated silica sol in example 7 and fumed aluminum oxide in place of the mixed silicon-aluminum oxide powder in example 8.

### Example 9 (Inventive): Redispersible Powder

A dispersion produced according to example 1 is used to generate an easily redispersible powder with the aid of a Mini Spray Dryer B-290 from BÜCHI Labortechnik GmbH using nitrogen as the hot gas medium: Stirring-in using a magnetic stirrer affords a d₅₀ of 155 nm, with a dissolver after 5 minutes at 2000 rpm a d₅₀ of 136 nm and with an ULTRA-TURRAX® T 25, IKA®-Werke GmbH & CO. KG after a minute at 9000 rpm a d₅₀ of 130 nm.

1-28. (canceled)

29. An aqueous dispersion comprising a surface-modified hydrophilic mixed oxide powder comprising silicon and aluminum, wherein:
a) the surface of particles in the powder has Si and Al atoms;
b) the surface modification comprises an Si atom bound to a hydrocarbon radical by a C atom; and
c) the carbon content of the surface-modified mixed oxide powder is 3-25% by weight.

30. The aqueous dispersion of claim 29, wherein the Si atom which is bound to the hydrocarbon radical by a C atom forms —Si—O—Al— bonds, the Al atom being a constituent of the particle surface.

31. The aqueous dispersion of claim 29, wherein the Al₂O₃/SiO₂ weight ratio in the surface-modified mixed oxide powder is 0.1:99.9-5:95.

32. The aqueous dispersion of claim 29, wherein the (Al₂O₃/SiO₂)_{surface}/(Al₂O₃/SiO₂)ₜₜₗ is 0.1-10.

33. The aqueous dispersion of claim 29, wherein the surface-modified mixed oxide powder is present predominantly or completely in the form of aggregated particles.

34. The aqueous dispersion of claim 29, wherein the surface-modified mixed oxide powder has a median particle diameter d₅₀ in the aqueous dispersion of 40-200 nm.

35. The aqueous dispersion of claim 29, wherein the hydrocarbon radical bonded to an Si atom by a C atom is interrupted by one or more heteroatoms.

36. The aqueous dispersion of claim 29, wherein the surface modification has the formula Si—(CH₂)ₙ—Yₘ—R, wherein Si is the Si atom bound to a hydrocarbon radical by a C atom and:
n=1, 2 or 3 and m=0 or 1;
Y═—(OCR¹R²—CR³R⁴)ₒ—, wherein o=1-30 and R¹, R², R³, R⁴=independently of one another H or CH₃; or —(OCR¹R²—CR³R⁴—CR⁵R⁶)ₚ—, wherein p=1-30 and R¹, R², R³, R⁴, R⁵, R⁶=independently of one another H or CH₃, —NHCH₂CH₂O—, —NH(CH₂)₂NH(CH₂)₂—, —NH(CH₂)₂NH(CH₂)₂—;
R is a radical which does not impart hydrophobic properties or is a mixture of the above mentioned radicals R and Y.

37. The aqueous dispersion of claim 29, wherein the proportion of water is 50-90% by weight and the proportion of surface-modified mixed oxide powder is 10-50% by weight.

38. The aqueous dispersion of claim 29, wherein the pH of the liquid phase of the aqueous dispersion is 8 to 12.

39. A process for producing the aqueous dispersion of claim 29, comprising:
a) dispersing in an aqueous solvent a mixed oxide powder comprising silicon and aluminum, which comprises Si atoms and Al atoms each bearing hydroxyl groups on the surface of particles in the powder; and
b) subsequently adding an agent for surface modification in which an Si atom is bound to a hydrocarbon radical by a C atom and the Si atom is further bound to one or more hydroxyl groups, alkoxy groups, halide groups or mixtures thereof;
c) allowing the mixture to react and optionally separating the hydrolysis product and adjusting pH;
wherein the agent for surface modification is added in such an amount as to obtain a carbon content of 3-25% by weight, taking account of the hydroxyl groups, alkoxy groups or halide groups eliminated in the hydrolysis.

40. The process of claim 39, wherein the mixed oxide powder comprising silicon and aluminum is introduced in the form of an aqueous dispersion.

41. The process of claim 39 wherein the mixture is reacted by adjusting the pH to 11 or higher, thermally treating the mixture at a temperature of 50-95° C. over a period of 1-30 minutes, and then optionally adjusting the pH to 8-10.

42. The process of claim 39, wherein a mixed silicon-aluminum oxide powder produced by pyrogenic means is employed.

43. The process of claim 39, wherein the agent for surface modification has the formula X₄₋ₐ[Si—(CH₂)ₙ—Yₘ—R]ₐ, where a=1, 2 or 3; n=1, 2 or 3; m=0 or 1; X═H, OH, OCH₃, OC₂H₅, OCH₂CH₂H₃, OCH(CH₃)₂; Cl, and R is a radical which does not impart hydrophobic properties or is a mixture of the above mentioned radicals R and Y.

44. The process of claim 43, wherein, in the case where m=1, R═—H, —CH₃, —C₂H₅, —OH, —OCH₃, —OC₂H₅, —C(═O)OCH₃, —C(═O)OC₂H₅, —O—C(═O)CH₃, —O—C(═O)CH₃, —O—C(═O)CH═CH₂, —O—C(═O)CH═CH(CH₃), —C(═O)CH₃, —C(═O)H, NH₂; or 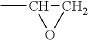
and in the case where m=0, the aforementioned radicals R are without —H, —CH₃, —C₂H₅.

45. The process of claim 39, wherein the agent for surface modification is selected from the group consisting of: (CH₃O)₃Si(CH₂)₃—OCH₃; (CH₃O)₃Si(CH₂)₃—(OCH₂CH₂)₃—OCH₃; (CH₃O)₃Si(CH₂)₃—(OCH₂CH₂)₆₋₉—OCH₃; (CH₃ O)₃Si(CH₂)₃—(OCH₂CH₂)₉₋₁₂—OCH₃; (CH₃O)₃Si(CH₂)₃—(OCH₂CH₂)₂₁₋₂₄—OCH₃; and (CH₃CH₂ O)₃Si(CH₂)₃—(OCH₂CH₂)₈₋₁₂OH.

46. The process of claim 39, wherein the agent for surface modification is selected from the group consisting of: (RO)₃Si—(CH₂)₃—NH₂; (RO)₃Si—(CH₂)₃—CH—CH₂—NH₂; (RO)₃Si—(CH₂)₃—NH—(CH₂)₂—NH₂; (RO)₃Si—(CH₂)₃—NH—(CH₂)₂NH(CH₂)—NH₂; (RO)₃Si—(CH₂)₃—N—[(CH₂)₂NH(CH₂)—NH₂]₂; and R═CH₃, C₂H₅.

47. The process of claim 39, wherein the agent for surface modification is an aqueous composition which bears organopolysiloxanes having glycidyl ether alkyl radicals, acryloyloxyalkyl radicals and/or methacryloyloxyalkyl radicals, with each silicon in the organopolysiloxane bearing a functional group.

48. A surface-modified mixed oxide powder comprising silicon and aluminum obtainable by:
a) dispersing in an aqueous solvent a mixed oxide powder comprising silicon and aluminum, which comprises Si atoms and Al atoms each bearing hydroxyl groups on the surface of particles in the powder; and
b) subsequently adding an agent for surface modification in which an Si atom is bound to a hydrocarbon radical by a C atom and the Si atom is further bound to one or more hydroxyl groups, alkoxy groups, halide groups or mixtures thereof;
c) allowing the mixture to react and optionally separating the hydrolysis product and adjusting pH.
